(19) United States
(12) Patent Application Publication
(10) Pub. No.: US 2008/0206196 A1
(43) Pub. Date: Aug. 28, 2008

(54) Differentiation of cord blood into neural like cells, and method to treat neurological condition patients

(76) Inventors: Calvin Cao, Tampa, FL (US); Lixian Jiang, Land O Lakes, FL (US)

Correspondence Address: Lixian Jiang, 2102 Camp Indianhead Road, Land O Lakes, FL, 34639, US

(21) Appl. No.: 11/710,817

(22) Filed: Feb. 26, 2007

(51) Int. Cl. C12N 5/06; A61K 39/00

(52) U.S. Cl. 424/93.1; 435/368; 435/374; 435/377

(57) ABSTRACT

The invention discloses a method of expansion and differentiation umbilical cord blood cells into neural-like cells in clinic grade, the cell preparation and herein the method for treating neurological condition in human.

### FIELD OF THE INVENTION

The present invention discloses a method to expand mononuclear portion cells of umbilical cord blood, and differentiate them into neural like cells for clinical use in treatment neurological conditions.

### BACKGROUND OF THE INVENTION

Human umbilical cord blood (HUCB) is a rich source of stem cells that have been used to reconstitute immune cells and blood lineage for the treatment of hematological diseases. The first clinical use of HUCB cells was in 1988 on a patient with Fanconi anemia. Since that time, more than 1000 transplants have been performed around the world in treatment of hematopoietic and genetic disorders including lymphoid and myeloid leukemia, Fanconi anemia, aplastic anemia, Hunter syndrome, Wiskott-Aldrich syndrome, beta-thalassemia, and neuroblastoma. Cord blood is the blood contained in the umbilical vein within the placental stump that is normally discarded after delivery of the neonate. Cord blood can be collected after clamping and cutting the umbilical cord, immediately after the birth of the baby. Compared to bone marrow, HUCB transplantation has less morbidity and mortality. Cord blood banks are being established in many countries including the United States and standard protocols for sample processing and storage developed.

The actual part of the cord blood that is used for cell transplantation is the “mononuclear fraction”, a fraction of the blood containing mainly mononuclear cells that is obtained by cell gradient separation techniques. Included in this fraction are the stem cells (HSC), which are multi-potential and can proliferate and differentiate into all lineages of haematopoietic cells. HSC cells are usually CD34+ positive cells, although CD34+ cells constitute a very heterogeneous cell population. The majority of CD34+ cells express both HLA-DR and CD38 antigens, while the most primitive HSC lack the expression of HLA-DR and CD38. CD34+ cells can further differentiate into three different progenitor populations. The first is the multipotent progenitors, or the colony forming unit—multipotential (CFU-Mix) progenitors. The second one is the myeloid progenitors, which characters as colony forming unit granulocyte-macrophage (CFU-GM). The third one is erythroid progenitors, and its colony is characterized as colony forming unit granulocyte-macrophage-erythroid (CFU-E) and burst forming unit-erythroid (BFU-E). CFU-MIX progenitors express low/undetectable levels of both CD45RA and CD71, and CFU-G/M progenitors are CD34⁺ CD45RA⁺ CD71^{lo} cells, while erythroid progenitors are marked with CD34⁺ CD45RA^{lo} CD71⁺. The CD34+ cell content in HUCB has been shown to be around 1 percent of the mononuclear fraction. One milliliter HUCB there are about 8,000 primitive erythroid progenitors (BFU-E), between 13,000 and 24,000 myeloid progenitors (colony-forming units-granulocyte/macrophage [CFU-GM]), and between 1,000 and 10,000 multipotent progenitors (CFU-Mix).

Another component of cord blood cells is the lymphocyte, which is comprised of T and B cells, and accounts for almost 41 percent of the mononuclear fraction of HUCB. T cells are defined by the expression of the CD3 molecules, which roughly are divided into CD4 or CD8 positive cells. CD4 positive T cells are around 55% of the T cell population, and CD8 positive cells account for the remaining 45%. The proportion of CD4 and CD8 reflects the maturity of T cells. In development, the ratio of CD4 to CD8 T cells progressively increases over time at least till subjects are adult. Actually, in the T cells population there are a small number of cells that express CD 16 and/or CD56 cells without co-expression of CD3. These cells are Natural Killer (NK) cells, and they are important against cancer for killing cells. In contrast, B cells are defined by the expression of CD19, a specific marker of B cell lineage, which starts to express from the differentiation of B cell progenitors, continues on pre-B cell, and all the way down to mature B cells. B cells are about 20% in HUCB, and decline in adulthood.

Monocytes are also component of the mononuclear faction, and they take the remaining portion of the HUCB mononuclear fractions. Monocytes are derived from committed myeloid progenitor cells, circulate in the blood and enter tissues to become resident tissue macrophage. Monocytes can be isolated by flow cytometry according to their cell surface antigens CD11b, CD18, CD14, and CD16. CD11b/CD18 are receptors on the surface of monocytes. They interact with intercellular adhesion molecule I on the endothelium and localize monocyte at the sites of infection. Once monocytes enter different tissues, they express certain enzymes, and can non-specifically take up particles such as colloidal carbon, and specific endocytic receptors. While dendritic cells (DC) are antigen presenting cells, and can activate naïve T cells. They account for a very small portion of HUCB. They can be generated in vitro not only from CD34+ haematopoietic stem cells in bone marrow or cord blood, but also from peripheral blood CD14+ monocytes with IL-4 and GM-CSF stimulation. Immature DC cells are CD1a positive cells, and when they get more mature, they express CD83, CD80 and CD86. These markers are also expressed by monocytes.

In summary, HUCB mononuclear fraction is a very heterogeneous population that is roughly divided into T cells, B cells and monocytes, and each subpopulation takes one third of the whole mononuclear fraction of HUCB. Stem cells as well as DC cells only account for 1-3% of this HUCB fraction.

Since human umbilical cord blood (HUCB) cells contain rich hematopoietic stem cells, it is possible for these cells to proliferate, differentiate into neural like cells and replace the cell loss caused by stroke. When HUCB cells were directly delivered into rodents subjected to MCAO, recipient animals showed an improved neurological and behavioral recovery compared with non-transplanted animals. Animals with permanent MCAO given 1,000,000 HUCB cell intrastriatally were significantly less active than nontransplanted animals during both dark and light phases of the light cycle, activity of transplanted animals was similar to their novel baseline behavior compared to sham controls. Further transplanted animals learned to stay on the platform in the passive avoidance task significantly more quickly than the animals with permanent occlusion and no transplant. In addition, transplanted animals had better behavioral recovery in elevated body swing test and step test compared to nontransplanted animals.

Scientists also found this functional improvement could be achieved with HUCB intravenous (i.v.) administration. For example, Chen infused HUCB via tail vein 24 hours or 7 days after transient MCAO. The animals with HUCB administered at 24 hours post stroke had improved performance on the rotarod test and modified neurological severity score (mNSS) compared to control animals, while animals treated with HUCB 7 days after MCAO only improved on the rotarod test but not the mNSS, suggesting the importance of early intervention. When intraparenchymal administration was compared to i.v. administration, both treatment routes promoted significant functional improvement compared with stroke only animals. The animals received HUCB learned faster than nontransplanted animals (p<0.05). Interestingly, after 2 months only i.v. injected animals maintained functional improvements, particularly in the Step test. From a practical stand point, even though intravenous administration had the same therapeutic effect as direct administration, it has the great advantage of being more easily delivered.

The dose effect of HUCB administration after MCAO animal was also studied. Significant reductions in spontaneous activity were observed in animals treated with 10⁶ or high doses of cord blood cells in comparison to media treated controls. Reductions in elevated body-swing bias and improvements in the Step test were optimal at 10⁷ cells and did not improve further at higher doses. However, the higher the dose of cells, the smaller the infarct size in the stroked animals.

Improved outcome was also observed when HUCB cells were administrated to animals that underwent hemorrhagic stroke. HUCB was delivered by intravenous infusion into animals with hemorrhagic injury induced by intrastriatal injections of collagenase. The animals were subjected a battery of neurological and behavioral tests at 1 day after injury, which was followed by the administration of umbilical cord blood. The functional recovery was tested at 6, and 13 day post HUCB infusion. HUCB significantly improved behavioral recovery in the Step test at day 6, and elevated body-swing test at day 13 after intravenous administration, while improved neurological scores were observed day 6 to day 13.

The function of HUCB cells was also evaluated in rats with spinal cord injury induced by hemicompression at T8/9 with calibrated aneurysm clip. One million HUCB cells were infused into animals through tail vein at the day 1 or day 5 following spinal cord injury. Spontaneous activities of animals were monitored with digital camera for 3 weeks, and the behaviors were scored by the standard developed by Basso, Beattie and Bresnehan. It was found that animal functional improvement was progressive with time duration in all spinal cord injury groups, while animals treated with HUCB at day 5 after spinal cord injury had a better functional improvement than animals received HUCB at day 1 after injury as well as the control group. No significant differences were observed between animals received HUCB at day 1 after injury and control group at all time points. Histological examination revealed that HUCB did migrate into the injury place, and there were more cells in animals received HUCB at day 5 after injury than animals received HUCB at day 1 after injury. The data suggested that the functional improvement at least partially depends on how many HUCB migrated into injury site.

Amyotrophic lateral sclerosis (ALS) is a severe CNS disease with diffuse motor neuron degeneration. There are no effective treatments for this disease at the moment. Recently, HUCB was delivered into transgenic ALS mice through intravenous administration, and the functional recovery was examined. Animals receiving HUCB had a longer life span compared to control animals. HUCB cells were found in the degenerating region of the brain and spinal cord, and some exhibited neural phenotypes. There, some HUCB cells are positively labeled with the antibodies against neural markers including Nestin, III Beta-Tubulin (TuJ1), and glial fibrillary acidic protein (GFAP). Animals receiving high dose HUCB have a significant longer life span than animals with low dose HUCB administration. Based on these promising effects of HUCB treatment of ALS mice, the clinical trials with hematopoietic stem cells are under way. Janson et al isolated CD34+ stem/progenitor cells from peripheral blood, and CD34+ cells are believed to be the key stem cell component in HUCB. The purified CD34+ cells were intrathecally injected into the peripheral-blood in three ALS patients. After 6-12 months, none of the patients reported side-effects, but no clinical efficacy was seen. Although the expected success was not achieved, there were at least no harmful effects to the patients. Further, since the total cases are too few for statistics analysis, it is too early to make a conclusion.

Previous studies demonstrated that cells from umbilical cord blood have the potentials to become a neural like cell, which could be used to treat neurological conditions for some chronicle patients.

### SUMMARY OF THE INVENTION

The invention describes an efficient method of generating a clinically significant quantity of neural like cells from umbilical cord blood. The cord blood is collected from pregnant mother when she gave birth after get her permission. The cord blood is screened from any infectious diseases, which include but not limited HIV, hepatitis B, hepatitis C and other kind of bacteria.

The red cells in the umbilical cord blood are removed with gradient separation techniques. The mononuclear portion of the cord blood cells are cultured in Dulbecco's Modified Eagles' Medium (DMEM) supplemented with fetal bovine serum (FBS) for 2-7 days to expand the cells. The expanded cells are cultured in DMEM medium in the presence of nerve growth factor (NGF) and retinoic acid (RA), which differentiate the cells into neural like cells.

### DETAILED DESCRIPTION

The present invention is providing a method of expansion a clinic grade stem cell solution with neural-like cell profile from umbilical cord blood, which would be benefit for treatment stroke, spinal cord injury, multiple sclerosis, amyotrophic lateral sclerosis, and other neurological diseases when transplanted into these patients who can not get improvement by conventional treatment. The said umbilical cord blood is collected from pregnant mom at birth with her permission.

The collected umbilical cord blood is separated into three parts A, B and C. The part A is processed for the tests of HIV, hepatitis B, C, bacteria and any other infectious disease to screen from all these diseases. The part B of collected cord blood is for cell expansion and differentiation as described following, when the tests of Part A cord blood are negative. The part C is stored in liquid nitrogen for further test for confirmation if the blood is contaminated with HIV, hepatitis B, C, bacteria and any other infectious disease or not.

The said part A blood of umbilical cord blood sample is processed to separate the mononuclear portion with gradient separation techniques. The part A of umbilical cord blood sample is diluted with an equal volume of D-hanks medium in the presence of heparin. Before process the separation procedure, the bottom chamber of Paque tube is filled with the Ficoll-Paque solution (1.077 g/ml) with 1.0 gauge syringe. Following that, the top of this chamber is placed with same amount of diluted umbilical cord blood. The cell solution is centrifuged at 1000 g for 10 minutes. The red cells then sit down at the bottom of the tube, and the serum is on the upper layer of the tube. Between these two layers, there is a white color layer, where the mononuclear cell portion is. The mononuclear portion is carefully harvested with aspiration to avoid mixing the cell solution.

The cells are suspended in 10 ml Dulbecco's Modified Eagles' Medium (DMEM) supplemented with 10% fetal bovine serum in the presence of Gentimacin (50 μl). Cells are centrifuged at 400× g for 15 min, the supernatant is removed and the pellet is re-suspended in 1 ml of the same medium. The cell viability ranged from 70% to 95% is determined by the trypan blue exclusive method. The cells are plated in 25 cm² plastic cell culture flask at the density of 100,000/ml. The cells are cultured at 37° C. in 5% CO2 in fully humid air for 2 days. The cells are gently shaken down to floating medium by hand, and transferred to a centrifuge tube. The solution is spanned at 1000 g for 10 minutes, and the supernatant is removed. And the pellet is re-suspended in the fresh medium as described before at the density of 200,000 cells per ml, and is cultured for another 2 days. Cells are changed one more time as the described above The cells at passage 3 are seeded in neuron growth medium (N5) supplemented with 10% horse serum, 1% FBS, transferrin (100 μg/ml), putrescine (60 μM), insulin (25 μg/ml), progesterone (0.02 μM), selenium (0.03 μM), all-trans-retinoic acid (0.5 μM) and brain derived neurotrophic factor (BDNF) at the concentration of 10 ng/ml. The cells are maintained in incubator at 37° C. in 5% CO2 in fully humid air for 5 days.

The cells are gently shaken floating in the medium, the cell solution is collected. The cell solution is centrifuged at 1000 g for 10 minutes, and the supernatant is removed. The cell pullet is re-suspended in DMEM supplemented with 10% FBS and adjusted to concentration at 500,000 cell per ml. The cell solution is separated into three parts, which is part a, b, c and d. Part “a” cell solution is tested of HIV, hepatitis B, C, bacteria and any other infectious disease. Part c is stored in liquid nitrogen for future use for determination the cell contamination of HIV, hepatitis B, hepatitis C, bacteria and other infectious diseases.

The part b of cell solution is suspended in 1 ml clear DMEM. The cells are incubated with primary antibodies including anti-Tuji1 (a neuron marker), anti-GFAP (astrocyte marker), and anti-CD11b (microglia marker). For one skill in the art, the primary antibodies could be any other specific antibody against neuron marker, astrocyte and microglia. For example, the antibody against NeuN is another neuron marker. The mixture is co-incubated for 15 minutes at room temperature. The cell solution is washed with clear DMEM three times, and re-suspended in 1 ml DMEM medium. The secondary antibody labeled with fluorescence or other dye against primary antibody is added into cell solution and incubated at room temperature for 15 minutes. Then the cell solution is washed by clear DMEM or PBS three times. The cell is re-suspended in DMEM medium, and the cell subpopulation is determined by flow cytometry analysis.

The cell composition for this clinic grade solution comprises the following cell population after culture: 50%-60% neurons, 38-49% astrocytes and less than 2% microglia cell. The solution should have very few cells from mononuclear portion of umbilical cord blood after this cell process.

The part d sample from umbilical cord will be used for human neurological condition which includes stroke, spinal cord injury, multiple sclerosis, and any other neurological diseases. The said part d sample will be either stored in liquid nitrogen or be used on the patients when it is ready to use.

For liquid nitrogen storage, the cells are adjusted to concentration at 1,000,000 cells per ml in DMEM supplemented with 10% FBS. For one ml cell solution, 55 μl dimethyl sulfoxide (DMSO) is added into the cell solution drop by drop, and swirl the cell solution at the same time to make DMSO evenly mixes with the cell solution. The cell solution is put into foam container, and put inside −80 degree freeze to make the temperature to drop 2-4 degree every hour for 24 hours. The cells are transferred into liquid nitrogen in the next day.

In one embodiment, the cell solution is stored in the liquid nitrogen as discussed above. It could be shipped to anywhere globally with liquid nitrogen or dry ice, which include hospital, clinic, tissue or blood bank, or transplantation center. This embodiment could allow the differentiated cells to store for more than 10 years, which is convenient for clinic use when it is demanded.

In another embodiment, the liquid stored cell solution could be thawed into clinic solution, which includes but not limit to PBS, glucose solution for cell transfusion or transplantation. At this step, the cell sample is sent to physician and thawed at water bath at 37 degree. The cells are then mixed with clinic solution alone or in combination of some other generic medication such as growth factor, nerve growth factor. Usually the dosage of these generic medications is at the maximal according to the pharmaceutical or FDA instruction.

In another embodiment, the cell solution could come directly from the cultured cells instead of the liquid nitrogen stored solution. The cell solution is shipped to the application site with sterile container in ice. The cells then are mixed with clinic solution as described before.

In one embodiment, neurological patients take MRI, CT scan, blood test and any other necessary tests depending on the patient condition before process the cell preparation treatment. Patients with other diseases combination such as tumor, coma and multiple organ failure are not suitable for this treatment protocol. Neurological test such as behavioral and recognition tests are performed to determine the injury sever of the nerve system. Patients with HIV and other sever infectious diseases are screened from this treatment as well.

The screened patients will be given 4 doses of cell solution, and each dose has at least 10 million alive cells inside. The 4 doses of cell solution will be administrated every 5 days. And the first shot is given through i.v. drop into at the speed of 3-5 drops every minute. Alternatively, the same amount of cells is delivered into patient through subdural injection, where the cells can directly move to the central nerve system through cerebrospinal fluid (CSF) flow. The whole process should be administrated to cause minimal damage when administration the cell preparation. The next following treatment protocol is 5 days later after the second cell preparation administration, and it follows the same treatment procedure as discussed for the first dose. The last dose is administrated within the last 5 days treatment frame through subdural injection.

After the cell preparation treatment, patients are examined with MRI, CT scan, blood test and other necessary tests, which should match the tests patients taken before. Behavioral and recognition tests are performed to determine the functional improvement in comparison the previous neurological test. Patients also follow up to see the functional improvement after they are discharged from hospital through phone call, in home visit or patient visiting hospital.

The following examples are the real person treated in our affiliated hospital as an illustration for this treatment protocol. All these hospitals are located outside United States where the local law allows the human research program in stem cell application. There is no intention to limit or narrow the scope of the present invention. The word using here should be given its broadest scope interpretation when it is used in its context.

### EXAMPLE 1

One patient from Denmark suffered from Multiple Sclerosis (MS) since 1983. The first symptoms occurred in 1982. The disease progression is active, and his current condition has score 4,5 on EDSS-scale on his left side of the body—leg and arm. His memory has deteriorated a lot, and when he gets tired, his voice is very low. He cannot walk without a cane, and has a lot of falls. The patient had a lung cancer surgery in 1989, which resulted in about ½ of his right lung removed. The patient voluntary participated in the research program of this cell preparation treatment in January 2007. Four doses of cell preparation were given to the patient through i.v. transfusion and subdural injection alternatively every 5 days. During this treatment, no immune suppression medication was used. After treatment, the patient has significant improvement that he can walk without cane. Further follow-up interview will continue.

### EXAMPLE 2

Another patient is from Nevada, USA suffered from optical nerve atrophy. The condition is caused by severe head trauma in October 2005 with subsequent coma over six weeks. Upon awakening, he was aware of severe impairment of vision which has fluctuated in severity but apparently gradually worsened to its present level. This patient voluntarily participated in our medical research with cell preparation treatment program in November 2006. Cell preparation was delivered into patient injury site through i.v drop and subdural injection. Total four doses of cell preparation were used. After treatment, the patient claimed he can see the picture and all color on the projector screen in the church from all the way in the back of the Church in Christmas day 2006. His vision went off and back again in January 2007. Further follow-up will be performed.

### EXAMPLE 3

Another patient is from New York, USA, suffered a stroke in February of 2006 that left him with left side paralysis. He voluntarily participated in the research program with cell preparation treatment in July, 2006. The patient was given a short treatment, where one dose cell preparation was administrated through i.v. injection, and the second one was given through subdural injection in the next 5 days. Two additional doses of cell preparation were administrated into patient through i.v drop and subdural injection alternatively every 5 days for 10 days, and at least 40 million qualified cells were used. The patient backed home after treatment. The patient had significant improvement that he can lift his foot, which was dropped before. One month later the patient walked to subway without a cane. The patient is still under follow-up.

What we claimed are:

1. A cell preparation for treatment neurological condition, comprising 50%-60% neurons, 38-49% astrocytes and less than 2% microglia cell;

2. A cell preparation of claim 1, wherein the said cell preparation is from the mononuclear portion of umbilical cord blood or bone marrow;

3. A cell preparation of claim 2, wherein the said mononuclear portion is expanded under Dulbecco's Modified Eagles' Medium (DMEM) supplemented with 10% fetal bovine serum in the presence of Gentimacin (50 μl/50 ml) for 3 passages.

4. A cell preparation of claim 3, wherein the expanded mononuclear cells are differentiated in neuron growth medium (N5) supplemented with 10% horse serum, 1% FBS, transferrin (100 μg/ml), putrescine (60 μM), insulin (25 μg/ml), progesterone (0.02 μM), selenium (0.03 μM), all-trans-retinoic acid (0.5 μM) and brain derived neurotrophic factor (BDNF) at the concentration of 10 ng/ml.

5. A method of expanding mononuclear portion of umbilical cord in step of;
a. providing cell culture medium comprising Dulbecco's Modified Eagles' Medium (DMEM) supplemented with 10% fetal bovine serum in the presence of Gentimacin (50 μl/50 ml);
b. culturing the cell in the said cell culture medium at 37 degree, humidity incubator;
c. changing the said medium every two days for at least 2 times;

6. A method of differentiation expanded mononuclear portion of umbilical cord into clinic grade neural-like cell solution in step of;
a. providing cell culture medium comprising neuron growth medium (N5) supplemented with 10% horse serum, 1% FBS, transferrin (100 μg/ml), putrescine (60 μM), insulin (25 μg/ml), progesterone (0.02 μM), selenium (0.03 μM), all-trans-retinoic acid (0.5 μM) and brain derived neurotrophic factor (BDNF) at the concentration of 10 ng/ml;
b. culturing the cell in the said cell culture medium at 37 degree, humidity incubator for at least 10 hours;

7. A method of storing cell solution for clinic use, in step of:
a. collecting the differentiated cells by vibration in claim 6,
b. centrifuging the cell solution at 1000 g for 10 minutes, and removing supernatant;
c. suspending the cell pellet in DMEM medium supplemented with 10% FBS at the concentration of 500,000 cell per ml to 20 million cells per ml;
d. freezing the suspended cell sample at a speed of 2-4 degree every hour for 24 hours, and storing the frozen cell solution for further use

8. A method of preparing cell solution for clinic use, in step of:
a. collecting the differentiated cells by vibration in claim 6,
b. centrifuging the cell solution at 1000 g for 10 minutes, and removing supernatant;
c. suspending the cell pellet in DMEM medium supplemented with 10% FBS at the concentration of 500,000 cell per ml to 20 million cells per ml;
d. Applying the prepared cell solution in neurological condition patients.

9. A method of treating neurological condition with the said cell preparation in claim 1, in step of:
a. combining the cell solution in at least 10 ml PBS or glucose solution;
b. transfusing one dose of said cell preparation in claim 1 into patient blood system through i.v. drop at first day;
c. subdural injecting one dose of said preparation in claim 1 into patient's cerebrospinal fluid (CSF) flow on the fifth day following first injection;
d. transfusing one dose of said cell preparation in claim 1 into patient blood system through i.v. drop on the tenth day following first injection;
e. subdural injecting one dose of said preparation in claim 1 into patient's cerebrospinal fluid (CSF) flow on the 15^{th} day following first injection;

10. A method of treating neurological condition in claim 9, wherein the method does not need immune suppression agent when the cell preparation is from umbilical cord blood;

11. A method of treating neurological condition in claim 9, wherein the method does not need the donor cells match the recipient immune system, when the cell preparation is from umbilical cord blood;
